(19) United States
(12) Patent Application Publication
(10) Pub. No.: US 2026/0207848 A1
(43) Pub. Date: Jul. 23, 2026

(54) Layer Bonding Glass Syringe

(71) Applicant: ATS Corporation, Cambridge (CA)

(72) Inventor: Roland LINDNER, Cambridge (CA)

(21) Appl. No.: 19/393,138

(22) Filed: Nov. 18, 2025

Related U.S. Application Data
(60) Provisional application No. 63/722,227, filed on Nov. 19, 2024.

(51) Int. Cl. A61M 5/31; A61M 5/34; B29C 35/08

(52) U.S. Cl. CPC A61M 5/3129; A61M 5/349; B29C 35/0805; A61M 2205/02; B29C 2035/0827

(57) ABSTRACT

A medical glass syringe includes a glass barrel and a needle. The glass barrel includes a needle hub, the needle is fixed in the needle hub with a first adhesive, and the exit end of the needle hub is provided with a second adhesive. A method of treating a medical glass syringe includes curing the first and second adhesives with radiation

### CROSS-REFERENCE TO RELATED APPLICATIONS

This application claims priority to U.S. Provisional Patent Application No. 63/722,227, filed on Nov. 19, 2024, the disclosure of which is hereby incorporated by reference herein in its entirety.

### FIELD OF THE INVENTION

This invention relates to a medical glass syringe and a method of treating a medical glass syringe.

### BACKGROUND OF THE INVENTION

In medical applications, syringes must be reliable, ensuring that the needle is firmly fixed to prevent detachment, leakage, or contamination of the drug inside. The process of attaching the needle typically involves adhesives, which must be carefully chosen and applied to meet the stringent requirements of medical use, including compatibility with drugs, resistance to environmental factors, and proper curing under manufacturing conditions.

One problem with conventional syringes is ensuring the secure and reliable attachment of the needle to the glass barrel while maintaining the integrity of the drug inside the syringe. Traditional methods can suffer from issues like improper adhesive curing, insufficient bond strength, or contamination risks due to interaction between the drug and the adhesive. Additionally, curing adhesives in a controlled manner can be challenging, especially when different parts of the syringe are exposed to varying levels of radiation during the curing process. Thus, there is a demand in the art for improved medical glass syringes.

### SUMMARY OF THE INVENTION

The present invention overcomes various deficiencies of the prior art. One solution utilizes a combination of adhesives with specific properties—such as differing viscosities, optical characteristics, and curing behaviors—tailored to optimize the syringe's performance. The invention also addresses the problem of ensuring that only certain adhesives come into contact with the drug, thereby maintaining the drug's purity and efficacy.

An aspect of the invention relates to a medical glass syringe, comprising a glass barrel and a needle, the glass barrel comprising a needle hub, wherein the needle is fixed by means of a first adhesive, and wherein the exit end of the needle hub is provided with a second adhesive. It is customary in the art that a needle is a slender, hollow, metal tube attached to a syringe, used for injecting fluids into or withdrawing fluids from a body. It typically has a sharp, pointed end for piercing skin and a hollow interior through which the fluid flows. A glass barrel is the main cylindrical body of the syringe made of glass, which holds and contains the medical drug or fluid to be injected. A glass barrel is generally transparent, allowing visual inspection of the contents and dosage, and is designed to be chemically inert to prevent interaction with the drug. The term interior space relates to the internal cavity within the glass barrel where the medical drug or fluid is stored. This space is isolated from the external environment and is in communication with the needle, allowing for the controlled dispensing of the contents through the needle. A needle hub is the component that connects the needle to the glass barrel of the syringe. It provides a secure interface for attaching the needle and can incorporate adhesives to bond the needle to the syringe. The exit end of the needle hub is the terminal part of the needle hub from which the needle protrudes. It is the point where the needle exits the hub and extends outward, e.g. for injection. The exit end may be sealed and reinforced with adhesives to provide a secure and leak-proof connection between the needle and the syringe.

In such a syringe, the use of two different adhesives ensures that the needle is securely attached to the syringe, minimizing the risk of detachment during use. The first adhesive firmly fixes the needle to the hub, while the second adhesive at the exit end provides an additional layer of security, potentially preventing leakage of medical fluids. This dual adhesive system can enhance the safety and reliability of the syringe. This way, the first adhesive can be chosen by providing an optimized compatibility with drugs inside the syringe, while the second adhesive can be chosen by providing optimized fixation of the needle without needing to take the drug compatibility of the second adhesive into account, as it is not in contact with the drug inside the syringe.

Further improvement can be achieved, when the second adhesive has generally no oxygen inhibiting properties and/or wherein the first adhesive has oxygen inhibiting properties. In such an embodiment, oxygen inhibition refers to the interaction between the adhesive and oxygen at the surface. When the adhesive with oxygen-inhibiting properties is exposed to air, oxygen can prevent the formation of long-chain polymerization: a process where molecules link together to form a strong, durable structure. Instead, only short-chain oligomers are formed. When the first adhesive comes into contact with oxygen, the oxygen binds to the free radicals that are necessary for long-chain polymerization, which is the process where molecules link together to form a strong and durable bond. Because of this, only short-chain oligomers are formed, which are shorter and less stable molecular chains. These oligomers cannot undergo any further light-stimulated reactions, leading to the formation of a gel-like, wet layer on the surface of the adhesive.

Further improvement is achieved, when the second adhesive has a lower viscosity than the first adhesive in uncured condition. In such an embodiment, the lower viscosity of the second adhesive allows it to flow more easily into small gaps and spaces at the exit end of the needle hub, ensuring a thorough and even application. This characteristic helps create a secure seal while avoiding excess adhesive buildup, which could interfere with the syringe's function. The higher viscosity of the first adhesive provides a stronger bond where it is most needed to hold the needle firmly in place. This combination of adhesives with different viscosities enhances the overall assembly process and ensures the syringe's reliability during medical procedures.

Further improvement is achieved when the first and the second adhesive are curable with radiation of the same wavelength, wherein the wavelength of the radiation is in the range 320 nm to 410 nm, preferably in the range 340 nm to 390 nm, most preferably around 365 nm. In such an embodiment, curing both adhesives with the same wavelength of radiation simplifies the manufacturing process, as only one type of curing equipment is needed. The specific wavelength range ensures effective curing without damaging the syringe materials. The preferred wavelength of for instance 365 nm is commonly used in UV curing processes. This uniform curing method enhances production speed and consistency, ensuring that the adhesives bond properly, resulting in a high-quality, durable syringe suitable for important medical applications.

Further improvement is achieved when the first and the second adhesive have the same optical properties when cured. In such an embodiment, using adhesives with the same optical properties ensures a uniform appearance of the syringe after curing. This uniformity is not only aesthetically preferred but also beneficial for quality control, as any inconsistencies in the bonding process would be more noticeable. Additionally, matching optical properties can prevent issues like light refraction or reflection at the adhesive joints, which could interfere with the syringe's function or inspection processes. Using adhesives with the same optical properties enhances both the visual quality and performance reliability of the syringe.

Further improvement is achieved when, the weight ratio between the first and the second adhesive is in the range of 0.2 to 1.8, preferably in the range of 0.6 to 1.4, most preferably 1.0, when uncured. In such an embodiment, setting the weight ratio of the adhesives ensures that the right amount of each adhesive is used to achieve optimal bonding and performance. The most preferred ratio of 1.0 indicates that equal amounts of both adhesives are used, which can simplify the manufacturing process and ensure consistency. By controlling this ratio, the syringe can maintain a balance between the strength provided by the first adhesive and the flexibility or flow properties of the second adhesive. This careful balance enhances the syringe's durability and functionality, making it suitable for medical applications that require precise and reliable performance.

Further improvement is achieved when the second adhesive forms a dome-shaped structure on the exit end, wherein the height of the dome-shaped structure is preferably in the range of 1 mm to 5 mm, more preferably in the range of 2 mm to 4 mm, and most preferably around 3 mm, and wherein the diameter of the dome-shaped structure is preferably in the range of 2 mm to 8 mm, more preferably in the range of 4 mm to 6 mm, and most preferably about 5 mm. In such an embodiment, the dome-shaped structure created by the second adhesive provides a smooth, protective cover over the exit end of the needle hub. The specified dimensions ensure that the dome is large enough to offer protection and secure sealing, yet small enough to avoid interfering with the syringe's use. This design helps to prevent contamination, reduce the risk of injury from the needle, and enhance the overall safety and usability of the syringe. The carefully controlled dimensions of the dome can ensure consistent manufacturing quality and reliability.

Further improvement is achieved when the shaft of the needle passes through the dome-shaped structure. In such an embodiment, having the needle shaft pass through the dome-shaped adhesive structure ensures that the needle is securely encased and stabilized at the exit point. This design can provide additional support and protection for the needle, reducing the likelihood of bending or breakage during use. The dome-shaped structure also acts as a seal, preventing any leakage of fluids around the needle and enhancing the overall safety and hygiene of the syringe.

Further improvement can be achieved when the first adhesive is LOCTITE® AA 3345 and the second adhesive is preferably LOCTITE® AA 3942. In such an embodiment, using LOCTITE® AA 3345 as the first adhesive provides strong bonding properties, ideal for securely fixing the needle to the hub; it is furthermore a well-known adhesive, which is approved for using in contact with medicines. LOCTITE® AA 3942, used as the second adhesive, is selected for its specific characteristics that suit the application, such as its flow properties and curing behavior. These adhesives are well-known for their reliability in medical applications, ensuring that the syringe components are bonded with high strength and durability.

Further improvement is achieved when the first adhesive is in communication with the interior space of the glass barrel, wherein the second adhesive is not in communication with the interior space of the glass barrel. In such an embodiment, having the first adhesive in communication with the interior space of the glass barrel ensures that it securely seals and bonds the needle to the syringe in a way that prevents fluid leakage from the inside. This is important for maintaining the sterility and integrity of the medication within the syringe. The second adhesive, not being in contact with the interior, is applied externally, likely serving as an additional seal or protective layer without risking contamination of the syringe's contents. This design optimizes the syringe's safety and performance, making it especially suitable for medical procedures that require precise fluid handling and strict hygiene standards.

Further improvement is achieved when the first adhesive is arranged close to the interior space of the glass barrel, such that a medical drug inside the medical syringe is in contact with the first adhesive and not with the second adhesive. In such an embodiment, the first adhesive's placement near the interior ensures that it directly interacts with the medical drug, providing a secure seal that keeps the drug contained without leakage. By keeping the second adhesive out of contact with the drug, the design minimizes any potential interactions between the drug and the adhesive materials, which could affect the drug's stability or efficacy. This arrangement is beneficial in preserving the purity and effectiveness of the medication.

Further improvement is achieved when the barrel and the needle hub are integrally formed with each other. In such an embodiment, integrating the barrel and the needle hub into a single unit eliminates the need for separate parts and additional bonding processes, reducing the risk of leakage or detachment. This design enhances the structural integrity of the syringe, making it more robust and reliable. The seamless connection between the barrel and the hub also improves the manufacturing process by simplifying assembly and reducing potential points of failure.

Further improvement is achieved when the first adhesive is arranged in the needle hub as to extend to the exit end of the needle hub, and as to be flush with the surface of the exit end; and wherein preferably the second adhesive completely covers the first adhesive at the surface of the exit end. In such an embodiment, the first adhesive extends to the exit end of the needle hub, providing a strong bond right up to the point where the needle exits the syringe. By being flush with the surface, it ensures a smooth and even application area for the second adhesive. The second adhesive, which completely covers the first adhesive, adds an extra layer of protection and sealing at the exit end. This dual-layer approach enhances the overall strength and durability of the syringe while minimizing any risk of fluid leakage. The coverage by the second adhesive also improves the syringe's resistance to environmental factors, making it effective in maintaining sterility and integrity during medical procedures.

Another aspect of the invention is a method of treating a medical glass syringe in accordance with any one of the preceding embodiments, comprising the curing of the first and the second adhesive by means of radiation. Such a method involves exposing the syringe to a specific type of radiation, such as UVA light, which triggers the adhesives to cure and securely bond the needle to the glass barrel. This curing process ensures that the adhesives achieve their full bonding strength, resulting in a durable and reliable syringe. Using radiation to cure both adhesives simultaneously can streamline the manufacturing process, improving efficiency and consistency. This method is advantageous in producing high-quality medical syringes that meet stringent safety and performance standards.

This method can be improved when the means of radiation does not comprise a mercury vapour lamp and wherein the means of radiation preferably comprises a LED, the LED preferably configured to emitting UVA radiation.

In one preferred embodiment, using an LED to emit UVA radiation provides a safer and more energy-efficient alternative to traditional mercury vapor lamps. LEDs are more environmentally friendly, as they do not contain hazardous materials like mercury. Additionally, LEDs offer more precise control over the wavelength and intensity of the radiation, which can improve the curing process by ensuring that the adhesives cure uniformly and effectively. This method enhances the production quality of the syringe, making it more reliable and safer for medical use, while also aligning with modern sustainability practices.

### BRIEF DESCRIPTION OF THE DRAWINGS

In the following, preferred embodiments of the disclosure are disclosed by reference to the accompanying figures.

FIG. 1 illustrates a medical glass syringe according to an embodiment of the invention in a side view;

FIG. 2 shows a medical glass syringe according to an embodiment of the invention when exposed to UVA radiation in a side view;

FIG. 3 shows a medical glass syringe according to an embodiment of the invention when only the first adhesive is applied in a schematic view; and

FIG. 4 illustrates the medical glass syringe of FIG. 3 with the second adhesive in a schematic view.

### DETAILED DESCRIPTION OF THE PREFERRED EMBODIMENTS

The subsequent sections provide a detailed description of the invention, referencing the accompanying illustrations for clarity. The descriptions represent examples only and are not intended to limit the invention's scope. Identical reference numerals across the figures and text denote the same components. The illustrations may not reflect actual size or scale; their dimensions, proportions, and depictions of elements might be enhanced for better understanding and visual convenience.

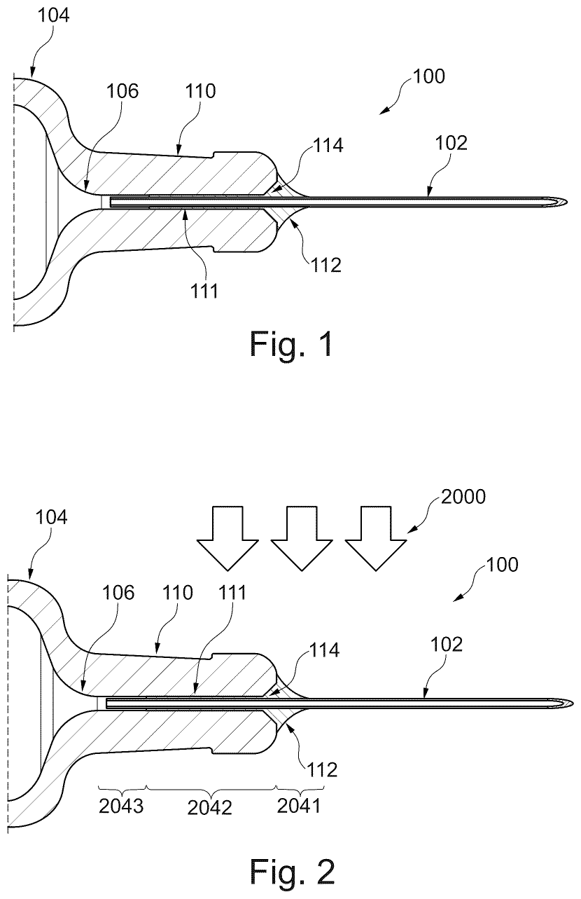

FIG. 1 illustrates a medical glass syringe according to an embodiment of the invention in a side view. The medical glass syringe 100 comprises a glass barrel 104 with a needle hub 110. The needle 102 is fixed in the needle hub 110 by means of a first adhesive 111, such that the first adhesive 111 is in communication with the interior space 106 of the medical glass syringe. The second adhesive 112 forms a dome-shaped structure at the exit of the needle hub 114 and additionally fixes the needle 102. This way, both the first and the second adhesive 111,112 fix the needle but only the first adhesive 111 can be in contact with a drug at the interior space 106 inside the glass barrel 104.

The use of both adhesives can ensure a robust and reliable fixation of the needle to the syringe. The first adhesive 111, being in contact with the drug, is selected for its compatibility with medical substances and its ability to prevent leaks into the interior space 106. The second adhesive 112, forming a dome-shaped structure at the needle's exit, provides additional security by covering and protecting the junction where the needle exits the hub. This dual adhesive approach enhances the syringe's safety and reliability, maintaining drug purity and preventing needle detachment.

The first and the second adhesives are preferably curable with radiation of the same wavelength. Preferably, the wavelength of the radiation is in the range 320 nm to 410 nm, more preferably in the range 340 nm to 390 nm, most preferably around 365 nm. Curing both adhesives with the same wavelength of radiation simplifies the manufacturing process, as only one type of curing equipment is needed. The specific wavelength range ensures effective curing without damaging the syringe materials. The preferred wavelength of, for instance, 365 nm is commonly used in UV curing processes. This uniform curing method enhances production speed and consistency, ensuring that the adhesives bond properly, resulting in a high-quality, durable syringe suitable for important medical applications.

The first and the second adhesives preferably have the same optical properties when cured. Using adhesives with the same optical properties ensures a uniform appearance of the syringe after curing. This uniformity is not only aesthetically preferred but also beneficial for quality control, as any inconsistencies in the bonding process would be more noticeable. Additionally, matching optical properties can prevent issues like light refraction or reflection at the adhesive joints, which could interfere with the syringe's function or inspection processes. Having the same optical properties enhances both the visual quality and performance reliability of the syringe.

The weight ratio between the first and the second adhesive is preferably in the range of 0.2 to 1.8, more preferably in the range of 0.6 to 1.4, most preferably 1.0, when uncured. Setting the weight ratio of the adhesives ensures that the right amount of each adhesive is used to achieve optimal bonding and performance. The most preferred ratio of 1.0 indicates that equal amounts of both adhesives are used, which can simplify the manufacturing process and ensure consistency. By controlling this ratio, the syringe can maintain a balance between the strength provided by the first adhesive and the flexibility or flow properties of the second adhesive. This careful balance enhances the syringe's durability and functionality, making it suitable for medical applications that require precise and reliable performance.

Preferably, the second adhesive has generally no oxygen inhibiting properties and/or the first adhesive has oxygen inhibiting properties, where oxygen inhibition refers to the interaction between the adhesive and oxygen at the surface. When the adhesive with oxygen-inhibiting properties is exposed to air, oxygen can prevent the formation of long-chain polymerization: a process where molecules link together to form a strong, durable structure. Instead, only short-chain oligomers are formed. When the first adhesive comes into contact with oxygen, the oxygen binds to the free radicals that are necessary for long-chain polymerization, which is the process where molecules link together to form a strong and durable bond. Because of this, only short-chain oligomers are formed, which are shorter and less stable molecular chains. These oligomers cannot undergo any further light-stimulated reactions, leading to the formation of a gel-like, wet layer on the surface of the adhesive.

Preferably, the second adhesive has a lower viscosity than the first adhesive in uncured condition. The lower viscosity of the second adhesive allows it to flow more easily into small gaps and spaces at the exit end of the needle hub, ensuring a thorough and even application. This characteristic helps create a secure seal while avoiding excess adhesive buildup, which could interfere with the syringe's function. The higher viscosity of the first adhesive provides a stronger bond where it is most needed to hold the needle firmly in place. This combination of adhesives with different viscosities enhances the overall assembly process and ensures the syringe's reliability during medical procedures.

FIG. 2 shows a medical glass syringe when exposed to UVA radiation in a side view. The medical glass syringe 100 comprises a glass barrel 104 with a needle hub 110. The needle 102 is fixed in the needle hub 110 by means of a first adhesive 111, such that the first adhesive 111 is in communication with the interior space 106 of the medical glass syringe. The second adhesive 112 forms a dome-shaped structure at the exit of the needle hub 114 and additionally fixes the needle 102. This way, both the first and the second adhesive 111,112 fix the needle but only the first adhesive 111 can be in contact with a drug at the interior space 106 inside the glass barrel 204. UVA radiation 2000 can penetrate the syringe completely, but the glass acts as a UV filter. UV curing should primarily focus on Zone C 2043, where the first adhesive 111 can be in direct contact with a drug inside the syringe. Since UVA radiation 2000 can pass through the second adhesive 112 in Zone A 2041 without a filtering effect of the glass, an imbalance in exposure occurs: When the first and the second adhesive 111,112 are the same, Zone C 2043 receives too little exposure, while Zone A 2041 tends to be overexposed.

By choosing differing first and second adhesives 111,112, this imbalance can be addressed. The first adhesive can be chosen for its ability to cure effectively under filtered UVA conditions in Zone C 2043, while the second adhesive can be selected to handle direct UVA exposure in Zone A 2041 without over-curing. This approach ensures that both adhesives cure properly, resulting in a syringe that is robust and reliable, without any over-and/or uncured adhesive residues.

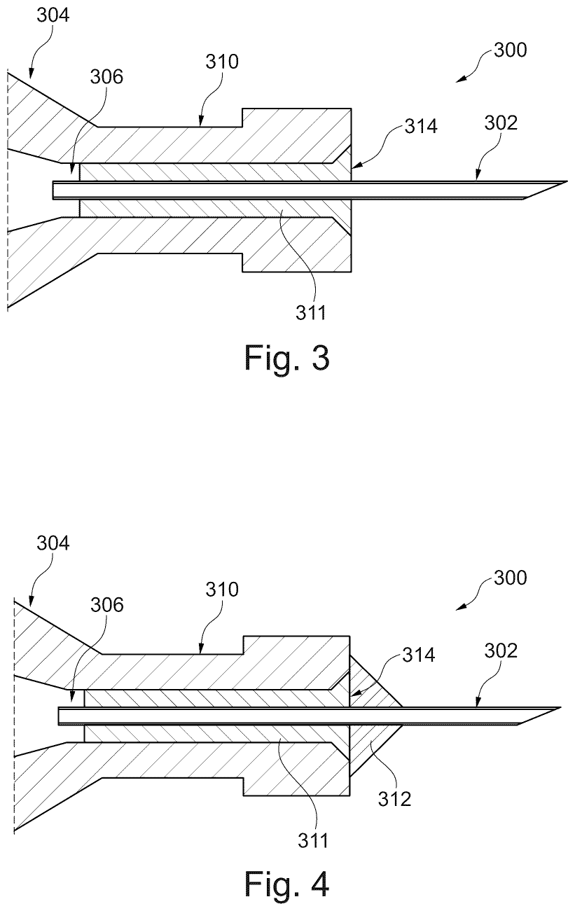

FIG. 3 shows a medical glass syringe when only the first adhesive is applied in a schematic view. The medical glass syringe 300 comprises a glass barrel 304 with a needle hub 310. The needle 302 is fixed in the needle hub 310 by means of a first adhesive 311, such that the first adhesive 311 is in communication with the interior space 306 of the medical glass syringe. The first adhesive 311 is arranged flush with the surface of the exit of the needle hub 314.

By positioning the first adhesive 311 flush with the surface of the needle hub's exit 314, the design minimizes any gaps or irregularities. This approach simplifies the syringe construction, making it easier to manufacture while still ensuring that the needle is firmly attached.

FIG. 4 illustrates the medical glass syringe of FIG. 3 with the second adhesive in a schematic view. FIG. 3 shows a medical glass syringe when only the first adhesive is applied in a schematic view. The medical glass syringe 300 comprises a glass barrel 304 with a needle hub 310. The needle 302 is fixed in the needle hub 310 by means of a first adhesive 311, such that the first adhesive 111 is in communication with the interior space 306 of the medical glass syringe. The first adhesive 311 is arranged flush with the surface of the exit of the needle hub 314. The second adhesive 312 is applied at the exit of the needle hub 314 and forms a dome-shaped structure, surrounding the needle 302 and completely covering the first adhesive 311 at the surface of the exit end 314. The height of the dome-shaped structure is preferably in the range of 1 mm to 5 mm, more preferably in the range of 2 mm to 4 mm, and most preferably around 3 mm. The diameter of the dome-shaped structure is preferably in the range of 2 mm to 8 mm, more preferably in the range of 4 mm to 6 mm, and most preferably about 5 mm.

The second adhesive 312 provides extra security and protection for the needle 302. The dome-like structure created by the second adhesive helps to further stabilize the needle at the exit point, reducing the risk of needle movement or detachment. This structure also acts as a protective barrier, potentially preventing contaminants from entering the syringe and ensuring that the needle remains securely in place during use.

It has been found that LOCTITE® AA 3345 is suitable for use as the first adhesive, and LOCTITE® AA 3942 is suitable for use as the second adhesive. LOCTITE® AA 3345 is a well-known adhesive, which is approved for use in contact with medicines. Using LOCTITE® AA 3345 as the first adhesive provides strong bonding properties, ideal for securely fixing the needle to the hub. LOCTITE® AA 3942, used as the second adhesive, is selected for its specific characteristics that suit the application, such as its flow properties and curing behavior. These adhesives are well-known for their reliability in medical applications, ensuring that the syringe components are bonded with high strength and durability.

The above-described embodiments are provided by way of example and are not intended to limit the scope of the invention. Persons of ordinary skill in the art will appreciate that various modifications and changes may be made without departing from the spirit and scope of the invention.

What is claimed:

1. A medical glass syringe, comprising:
a needle; and
a glass barrel including a needle hub;
wherein the needle is fixed in the needle hub by means of a first adhesive, and wherein an exit end of the needle hub is provided with a second adhesive.

2. A medical glass syringe according to claim 1, wherein the second adhesive has generally no oxygen inhibiting properties or the first adhesive has oxygen inhibiting properties.

3. A medical glass syringe according to claim 1, wherein the second adhesive has generally no oxygen inhibiting properties and the first adhesive has oxygen inhibiting properties.

4. A medical glass syringe according to claim 1, wherein, when uncured, the second adhesive has a lower viscosity than the first adhesive.

5. A medical glass syringe according to claim 1, wherein the first and the second adhesives are curable with radiation of the same wavelength.

6. A medical glass syringe according to claim 5, wherein the wavelength of the radiation is in the range 320 nm to 410 nm.

7. A medical glass syringe according to claim 5, wherein the wavelength of the radiation is in the range 340 nm to 390 nm.

8. A medical glass syringe according to claim 5, wherein the wavelength of the radiation is 365 nm.

9. A medical glass syringe according to claim 1, wherein the first and the second adhesives have the same optical properties when cured.

10. A medical glass syringe according to claim 1, wherein, when uncured, a weight ratio between the first and the second adhesives is in the range of 0.2 to 1.8.

11. A medical glass syringe according to claim 10, wherein the weight ratio between the first and second adhesives is in the range 0.6 to 1.4.

12. A medical glass syringe according to claim 10, wherein the weight ratio between the first and second adhesives is 1.0.

13. A medical glass syringe according to claim 1, wherein the second adhesive forms a dome-shaped structure on the exit end and wherein a height of the dome-shaped structure is in the range of 1 mm to 5 mm, and wherein a diameter of the dome-shaped structure is in the range of 2 mm to 8 mm.

14. A medical glass syringe according to claim 13, wherein a shaft of the needle passes through the dome-shaped structure.

15. A medical glass syringe according to claim 1, wherein the first adhesive is in communication with an interior space of the glass barrel and wherein the second adhesive is not in communication with the interior space of the glass barrel.

16. A medical glass syringe according to claim 1, wherein the first adhesive is arranged close to an interior space of the glass barrel, such that a medical drug inside the medical glass syringe is in contact with the first adhesive and not with the second adhesive.

17. A medical glass syringe according to claim 1, wherein the glass barrel and the needle hub are integrally formed with each other.

18. A medical glass syringe according to claim 1, wherein the first adhesive is arranged in the needle hub so as to extend to an exit end of the needle hub, and so as to be flush with a surface of the exit end; and
wherein the second adhesive completely covers the first adhesive at the surface of the exit end.

19. A method of treating a medical glass syringe comprising the steps of:
fixing a needle to a needle hub of a glass barrel of the medical glass syringe by means of a first adhesive;
providing an exit end of the needle hub with a second adhesive; and
curing the first and the second adhesives by means of radiation.

20. A method according to claim 19, wherein the means of radiation does not comprise a mercury vapour lamp and wherein the means of radiation comprises an LED configured to emit UVA radiation.
